(19) United States
(12) Patent Application Publication
(10) Pub. No.: US 2025/0250591 A1
(43) Pub. Date: Aug. 7, 2025

(54) GERMACRENE SYNTHASE SgTPS7 PROTEIN, CODING GENE AND USE IN SYNTHESIS OF GERMACRENE

(71) Applicant: Research Institute of Tropical Forestry, Chinese Academy of Forestry, Guangzhou (CN)

(72) Inventors: Niu YU, Guangzhou (CN); Mingliang DONG, Guangzhou (CN); Jinchang YANG, Guangzhou (CN)

(21) Appl. No.: 19/186,618

(22) Filed: Apr. 23, 2025

(30) Foreign Application Priority Data
Apr. 24, 2024 (CN) 202410495340.X

(51) Int. Cl. C12P 5/00; C12N 9/88

(52) U.S. Cl. CPC C12P 5/002; C12N 9/88; C12Y 402/03075

(57) ABSTRACT

The present disclosure discloses a germacrene synthase SgTPS7 protein, a coding gene and a use in synthesizing germacrene, relating to the technical field of germacrene synthesis of Sindora glabra Merr. ex de Wit. The present disclosure isolates a gene of germacrene synthase D from the genome of Sindora glabra Mer. ex de Wit. The gene encodes the protein germacrene synthase D which can catalyze the production of germacrene and can be used for the production and application of germacrene.

### SEQUENCE LISTING

The sequence listing is submitted as an XML file filed via EFS-Web, with a file name of “Sequence_Listing.XML”, a creation date of Apr. 19, 2025, and a size of 9,796 bytes. The sequence Listing filed via EFS-Web is a part of the specification and is incorporated in its entirety by reference herein.

### CROSS REFERENCE TO RELATED APPLICATIONS

The present disclosure claims the priority to the Chinese patent application with the filing No. 202410495340X, entitled “GERMACRENE SYNTHASE SgTPS7 PROTEIN, CODING GENE AND USE IN SYNTHESIS OF GERMACRENE” and filed on Apr. 24, 2024 with the Chinese Patent Office, the contents of which are incorporated herein by reference in their entirety.

### TECHNICAL FIELD

The present disclosure relates to the technical field of synthesis of germacrene of Sindora glabra Merr. ex de Wit, and more specifically to a germacrene synthase SgTPS7 protein, a coding gene and a use in synthesis of germacrene.

### BACKGROUND ART

Germacrene, a major member of the aromatic carbohydrate sesquiterpenes, has important antibacterial and insecticidal functions and is also an important aromatic component of essential oils. Germacrene contains five isomers, including germacrenes A-E, among which germacrene A and germacrene D are more widely distributed.

Sindora glabra Merr ex de Wit is a tall arbor tree of the Genus Sindora in the Family Caesalpiniaceae. The stems of Sindora glabra Merr. ex de Wit contain a large amount of sesquiterpenoid resin oils of more than 20 kinds, where the proportion of germacrene D reaches about 2.5%. Currently, germacrene is mainly extracted from plant tissues, which greatly limits the extraction yield and purity. The use of genetic engineering methods to produce and synthesize germacrene using Escherichia coli provides an effective solution for the mass production of germacrene.

Therefore, how to provide a gene and protein encoding germacrene synthase is a problem to be urgently solved by a person skilled in the art.

### SUMMARY

In view of this, the present disclosure isolates a gene of germacrene synthase D from the genome of Sindora glabra Merr ex de Wit. The gene encodes the protein germacrene synthase D, which can catalyze the production of germacrene and can be used for the production and application of germacrene.

In order to achieve the above objects, the present disclosure adopts the following technical solution.

Germacrene synthase SgTPS7 protein, the amino acid sequence of the protein is as shown in SEQ ID NO. 1.

Further, the present disclosure further seeks to protect a gene, which encodes the protein sequence as shown in SEQ ID NO. 1.

Preferably, the gene is the SgTPS7 gene, and the nucleotide sequence is as shown in SEQ ID NO. 2.

Further, the present disclosure further seeks to protect the use of germacrene synthase SgTPS7 protein or a coding gene of the SgTPS7 protein in catalyzing FPP or GPP to form germacrene.

Further, the present disclosure further seeks to protect an expression element of germacrene synthase SgTPS7, where the expression element is a pET30a-SgTPS7 recombinant plasmid.

Further, the present disclosure further seeks to protect a method for synthesizing germacrene D, including steps of: transferring the pET30a-SgTPS7 recombinant plasmid into competent cells of Escherichia coli, culturing, centrifuging, resuspending and purifying to obtain the germacrene synthase SgTPS7 protein, and then catalyzing FPP or GPP using the SgTPS7 protein to obtain germacrene D.

It can be seen from the above technical solution that compared with the prior art, the present disclosure discloses a germacrene synthase SgTPS7 gene, and the SgTPS7 protein encoded thereby can catalyze FPP or GPP as a substrate to obtain the germacrene D, thereby realizing the mass production of the germacrene D.

### BRIEF DESCRIPTION OF DRAWINGS

In order to more clearly illustrate the technical solutions of the embodiments of the present disclosure or in the prior art, the drawings required to be used the description of the embodiments or the prior art will be briefly introduced below. Apparently, the drawings described below are only embodiments of the present disclosure. For a person ordinarily skilled in the art, other drawings may be obtained based on the provided drawings without paying creative work.

FIG. 1 is an agarose gel electrophoretogram of the PCR product of a SgTPS7 gene;

FIG. 2 is an enzyme digestion electrophoretogram of a pET30a-SgTPS7 vector; 1: Plasmid DNA; 2: digested with NdeI/HindIII; 3: DNA Marker;

FIG. 3 is a structural view of the pET30a-SgTPS7 vector;

FIG. 4 is a view showing expression of TPS7 protein in BL21 (DE3) analyzed by SDS-PAGE; M: SDS-PAGE Protein Marker; 0: control (without IPTG); 1: 15° C. induction for 16 h; 2: 37° C. induction for 16 h;

FIG. 5 is a view showing the solubility result of TPS7 protein analyzed by SDS-PAGE; M: SDS-PAGE Protein Marker; 1: supernatant after whole bacteria lysis; 2: precipitate after whole bacteria lysis;

FIG. 6 shows the purification result of TPS7 protein supernatant analyzed by SDS-PAGE; M: SDS-PAGE Protein Marker; 1: flow-through after incubation of supernatant with Ni-IDA; 2-4: elution fractions of 100 mM Imidazole; 5-6: elution fractions of 300 mM Imidazole;

FIG. 7 shows the purity of TPS7 protein analyzed by SDS-PAGE; 1: BSA (2.00 μg); 2: TPS7 protein (1.00 μg); M1: SDS-PAGE Marker; and

FIG. 8 is view showing the catalysate result of sesquiterpenoid synthase SgTPS7 of Sindora glabra Merr ex de Wit detected by GC-MS.

### DETAILED DESCRIPTION OF EMBODIMENTS

The technical solutions in embodiments of the present disclosure will be clearly and completely described below in conjunction with drawings in the embodiments of the present disclosure. Apparently, the described embodiments are only some, not all of the embodiments of the present disclosure. Based on the embodiments of the present disclosure, all other embodiments obtained by a person ordinarily skilled in the art without creative work shall fall within the scope of protection of the present disclosure.

### Example 1 Cloning of Germacrene Synthase D Gene SgTPS7 of Sindora glabra Merr. Ex De Wit

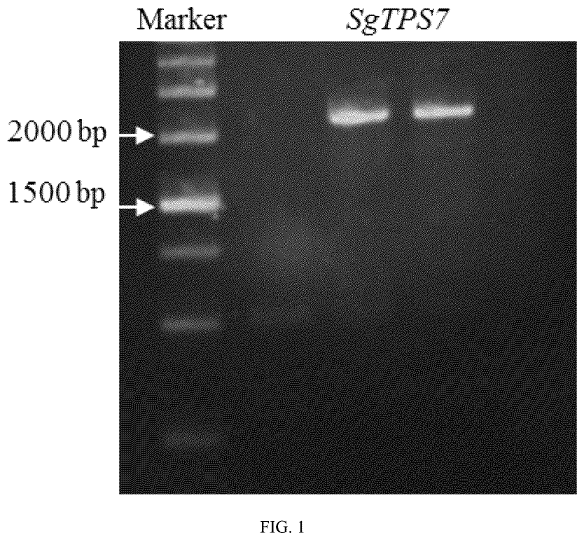

With the stem tissue of Sindora glabra Merr. ex de Wit used as a material, the total RNA of roots, stems and leaves of Sindora glabra Merr. ex de Wit was extracted by using an RNA kit (TIANGEN, DP441), and the extracted RNA was reverse transcribed into cDNA using a reverse transcription kit (TARAKA, RR470). PCR amplification was performed using a high-fidelity enzyme (TAKARA, R045A), with the cDNA used as a template. The amplified product was detected by 1.2% agarose gel electrophoresis, as shown in FIG. 1, and then the target band was excised, purified and recovered (TIANGEN, DP214), connected to the pUCm-T vector, transformed into competent cells of Escherichia coli Trans-T1, and screened for positive clone colony. After PCR detection, the bacterial solution was sent to Sangon Biotech (Shanghai) Co., Ltd. for sequencing. The recombinant plasmid was extracted from the sequencing-verified sample.

The nucleotide of the SgTPS7 gene is as shown in SEQ ID NO. 2, and the encoded protein is as shown in SEQ ID NO. 1.

>SgTPS7
ATGACTAGATCCACAGCAGGCTTCACTCCTAGTGTTTGGGGAAATCAGTT
CCTTTCCGTTGCTTCCAGCCCCTCGATGAAGAACAAAAGAGTGGAAATTC
ATCAATATTTACAGAATCTGAAACAACAACTGGAGAGACAGCTAAAAAGT
GTTGAAGAGCCCTCTGAAAAATTAAACTTGATCGATACCATGCAACGTTT
AGGAGTGTCTTACCATTTTCAAAGTGAGATCGAAGAATCATTAAAACATC
TGCATAAGAATCCTCCTCCTTCCTGGAATGCCAAAGATATCGATTCTCAT
CTTTTGGGCATTGCTCTTTGGTTTCGGCTGCTTAGACAACAAGGCTATTA
CGTATCATGCGATATATTTAACAAATTTAAAGATGAGAAAGGTGATTTCA
AGACAACATTGATCAATGATGTGGAAGGAATGCTAGCCTTATATGAAGCT
GCATATCTCGGTATCCGTGGAGAAGAAATTCTTGATCAAATGCTAGAATT
TACAACGTCTTACCTTAAATCAAGGTTGAATGACATGACACCCTACCTTC
AAGAAAGAACAAATCGTGCTTTGTACTGTCCTATACACAAAGGCTTACCA
AAAATTGAGACCAGATATTACATTTCTATGTATTCAAAAAAGGATTCTCG
GAATGATCTGCTACTAGAATTTGCGATACTAGATTTCAATATCTTGCAAC
AACAATATCAGAAGGAATTAAGCTACATCACAGAGTGGTTTAAAAAATTA
GATTTCGTGAGCAAGGTTCCTTACACCAGAGACAGAATTGTTGAAGGCTA
CTTTTGGCCTTTGGGAGCATACTTTGAGAAGCAATATAGCAAAGGAAGAA
GAATTGTGGCCAAAATTATTGGTGCTTTTTCATCTTTGGATGATACTTAT
GATAACTTCGGCACAGTGGATGAACTCAATGTCTTTACTGAAGCAATTAT
GAGATGGGATATTAATCTAGTAGCTTCTCTCCCTGAATCCATGAAAGTGG
TATTTGAAAATATTTTAAATTTGTTGATTGAAATAGAGTTGTTAACCGAA
GAGGACGAAATATCCTTTGCTGTTGAATATGTTAAACAAGGGATTCAAGG
TTTAGTGACAGGATACATGCTTGAAGCTGAGTGGAGGGGCAGAGGCTACA
TACCAACATATGAGGACTACATAGCGAATGGAATTTGGACCGCTGGTTAC
CCAGCCCTTGAAATAATATCCTTACTTGGTCTCGGAAATATCGCAACCCG
AGAGGTGTTTGATTGGATTTCTAGTATGCCTAAAATTGTTAGAGCTTCAG
GAATTGTGGGCAGAATAGGAAATGATTTGGCTTCTCATAAGAGGGAACAA
AATAGCGGACATGTTGCAACTTCAGTTGAGTGTTACATGAAGCAATATGG
GATTTCAGAAGATGAAGCCTATAAACTACTACTTAAGGAGATAGAGAATG
CATGGAAAGATCTTAACGAAGAGTATATGAAGCCAAATGGTATACCAAAG
GTGGTACTTGAGCGTGTACGGAATTTCTCGCGTGCAAGTGAGTTTTTCTA
TGGTCGCTTTGTTGATAATTACACAAATGGAGAGAGGCTGAAGGATCACA
TTGCTGCACTTTTTCTGGATCCCATAGCAATTGATCACAATAAATAG,
as shown in SEQ ID NO. 2.
>SgTPS7
MTRSTAGFTPSVWGNQFLSVASSPSMKNKRVEIHQYLQNLKQQLERQLKS
VEEPSEKLNLIDTMQRLGVSYHFQSEIEESLKHLHKNPPPSWNAKDIDSH
LLGIALWFRLLRQQGYYVSCDIFNKFKDEKGDFKTTLINDVEGMLALYEA
AYLGIRGEEILDQMLEFTTSYLKSRLNDMTPYLQERTNRALYCPIHKGLP
KIETRYYISMYSKKDSRNDLLLEFAILDFNILQQQYQKELSYITEWFKKL
DFVSKVPYTRDRIVEGYFWPLGAYFEKQYSKGRRIVAKIIGAFSSLDDTY
DNFGTVDELNVFTEAIMRWDINLVASLPESMKVVFENILNLLIEIELLTE
EDEISFAVEYVKQGIQGLVTGYMLEAEWRGRGYIPTYEDYIANGIWTAGY
PALEIISLLGLGNIATREVFDWISSMPKIVRASGIVGRIGNDLASHKREQ
NSGHVATSVECYMKQYGISEDEAYKLLLKEIENAWKDLNEEYMKPNGIPK
VVLERVRNFSRASEFFYGRFVDNYTNGERLKDHIAALFLDPIAIDHNK*,
as shown in SEQ ID NO. 1.

### Example 2 Construction of Prokaryotic Expression Vector of Germacrene Synthase D Gene SgTPS7 of Sindora glabra Merr. Ex De Wit

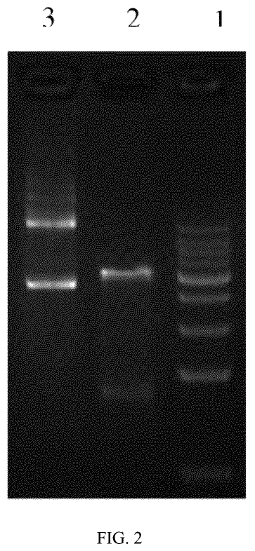

The amino acid sequence of SgTPS7 protein was optimized using the codon optimization software MaxCodon™ Optimization Program (V13), SgTPS7 gene was inserted into the expression vector pET30a by using whole gene synthesis and through restriction enzyme digestion sites NdeI and HindIII, and the accuracy of the final expression vector was confirmed by an enzyme digestion method and sequencing, as shown in FIG. 2.

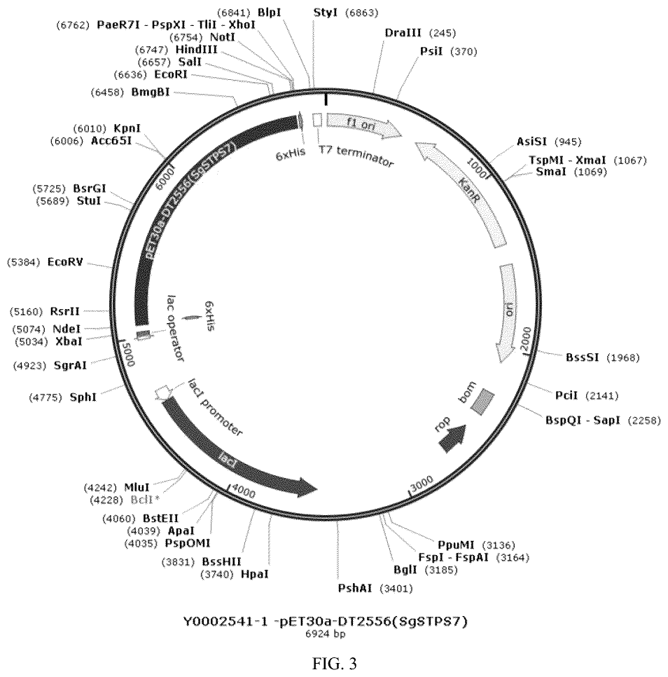

The structural view of the pET30a-SgTPS7 vector is as shown in FIG. 3.

The base sequence of SgTPS7 fusion protein is as follows:

>His-SgTPS7
CATATGCATCATCATCATCATCACGGTCGTCCGACCGCAAATTTTAGTCC
GAGCGTTTGGGGCAACCAGTTTTTATCTATTGCGAGCGGTCCGCTGCTGA
AAAACAAAGAAGCGGAGATCCACCAGCATCTGCTGAACCTGAAAGAACAG
GTCCGCAAACAGCTGAAAAACGGCGTTGAAGAACCGAGCGAGAAACTGAA
CCTGATCGATACCATCCAACGTCTGGGCGTTAGCCATCATTTCGAACGCG
AAATCGAAGAGAGCCTGAAACATCTGCATAAAAACCCGCCGAGTAGTTGG
AACGCAGAAGATATCGACGCGCATCTGCTGTCTATCAGTCTGTGGTTTCG
TCTGTTACGTCAGCAGGGCTATTACGTTAGCTGCGACGTTTTCGACAAAT
TCAAAGACGACAAAGGCATGTTCAAAACCACCCTGATTGACGACGTCGAA
GGTATGTTAGCGCTGTACGAAGCAACCTATCTGGGTATTCGCGGCGAAGA
AATCCTGGATCAGGTTCTGGAGTTCACCATGTTCCACCTGAAAAGCCGTC
TGGAAGGCATGACCCCGTATTTACGCGAACGCGCAGATCGCGCATTATAT
TGTCCGATCAACAAAGGCCTGCCGCGTATTGAAACCCGCTACTTCATCAG
CATGTACAGCAAAAAAGACAGCCGTAACGATCTGCTGCTGGAATTTGCAA
TGCTGGACTTTAACATCCTGCAGCAGCAGTACCAGAAAGAACTGTCTCAC
CTGAGCGAGTGGTACAAAAAACTGGACTTCGTCAGCAAAGTCCCGTATAC
CCGCGATCGTATTGTCGAAGGCTATTTCTGGCCGTTAGGCGCATATTTCG
AGAACCAGTACAACAAAGGCCGCATCATCGTCAGCAAACTGATCCTGGTT
CTGACCGCATTAGACGATACCTACGACGCATACGGTACCGTTGACGAACT
GAAACTGTTCACCGAGGCGATTAAACGCTGGGACATCAACATGGTCGCTT
CTTTACCGGAGTGCATGAAAGTCGTCTTTCAGGCGATCCTGGACTTACTG
GACGAAATGGAACTGCTGACCGAAGCTGACGGTATTAGCTGCTTCGTCGA
ATACGTTAAACCGGCACTGCAGGATCTGGCTAAAAGCTATCTGCTGGAAG
CAGAGTGGCGCGATAAAGGTTACATCCCGACCTACGAAGAATATATCGCG
AACGGCGTTTTTAGCTGCGGTTATCCGGCCGTTGAAATGGCAAGTCTGCT
GGGTCTGGGCAAAATTGCGACCAAAGAAGTCTTCGATTGGATCAGCAACG
TTCCGAAAATTGTTCGCGCGTCTAGCATCATGTGCCGTTTAACCGACGAT
CTGGCAAGCCACAAATTCGAACAGAACCGCGAACACGTCGGTTCCGCTAT
CGAGTGCTACATGAAACAGTACGGCGTTAGCGAAGAAGAAGCCTATAAAA
TGCTGCTGAAAGAGATCGAGAACGGCTGGAAAGACCTGAACGAAGAATAT
ATGAAACCGAACGGCGTTCCGAAAGTTGTACTGAAATGCGTCCTGAACTT
CAGCCGCGTTATTGAATTCCTGTACGGCCACTTCGTCGACAAATACACCA
ACGCGGAAATGCTGAAAGACCACATCGCGAGCCTGTTTGTTGATCCGATT
GCCATTGACGTCAACAAATAATGAAAGCTTG, as shown in
SEQ ID NO. 3.

The sequence of SgTPS7 fusion protein is as follows:

>His-SgTPS7
MHHHHHHTRSTAGFTPSVWGNQFLSVASSPSMKNKRVEIHQYLQNLKQQL
ERQLKSVEEPSEKLNLIDTMQRLGVSYHFQSEIEESLKHLHKNPPPSWNA
KDIDSHLLGIALWFRLLRQQGYYVSCDIFNKFKDEKGDFKTTLINDVEGM
LALYEAAYLGIRGEEILDQMLEFTTSYLKSRLNDMTPYLQERTNRALYCP
IHKGLPKIETRYYISMYSKKDSRNDLLLEFAILDFNILQQQYQKELSYIT
EWFKKLDFVSKVPYTRDRIVEGYFWPLGAYFEKQYSKGRRIVAKIIGAFS
SLDDTYDNFGTVDELNVFTEAIMRWDINLVASLPESMKVV
FENILNLLIE IELLTEEDEISFAVEYVKQG
IQGLVTGYMLEAEWRGRGYI PTYEDYIANG IWTAGYPALE
IISLLGLGNIATREVFDWIS SMPKIVRASG IVGRIGNDLA
SHKREQNSGHVATSVECYMKQYGISEDEAYKLLLKEIENAWKDLNEEYMK
PNPGIPKV VLE RVRNFSRASE
FFYGRFVDNYTNGERLKDHIAALFLDPIAI DHNK, as shown in
SEQ ID NO. 4.

### Example 3 Purification of Germacrene Synthase D Gene SgTPS7 Protein of Sindora glabra Merr. Ex De Wit

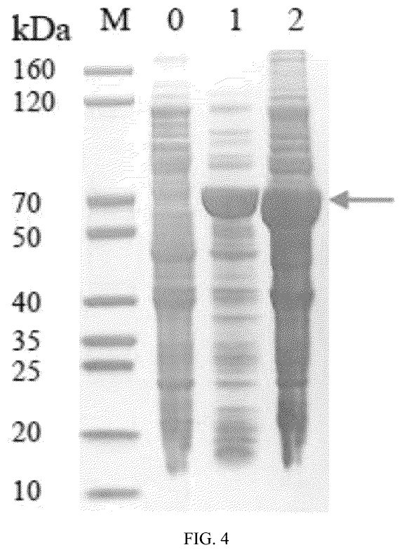

The constructed plasmid containing TPS7 gene was transformed into BL21(DE3) competent cells, then evenly spread on an LB plate (containing 50 μg/mL kanamycin sulfate), and then invertedly placed in a 37° C. incubator overnight. Monoclones were selected from the transformed plate, and inoculated into 4 mL of LB medium (containing 50 μg/mL kanamycin sulfate), and then cultured until OD₆₀₀ was 0.5-0.8, and then IPTG with a final concentration of 0.2 mM was added to the liquid culture medium in a test tube, and then placed at 15° C. and 37° C. respectively to induce expression. The induced liquid culture medium was collected and centrifuged at 12,000 rpm for 5 min, added with PBS solution after removing the supernatant to resuspend the precipitate, and finally added with SDS-PAGE loading buffer. The sample was heated at 100° C. for 10 min, and then centrifuged, and the supernatant was collected for electrophoresis. Electrophoresis was performed at a constant voltage of 160 V until the bromophenol blue band migrated to 1 cm from the bottom of the gel. The gel was taken out and stained and destained using a protein gel rapid processing system. The results are shown in FIG. 4. The arrow indicates the SgTPS7 protein.

### Example 4 Scale-Up Culturing of SgTPS7 Protein Encoded by Germacrene Synthase D Gene of Sindora glabra Merr Ex De Wit

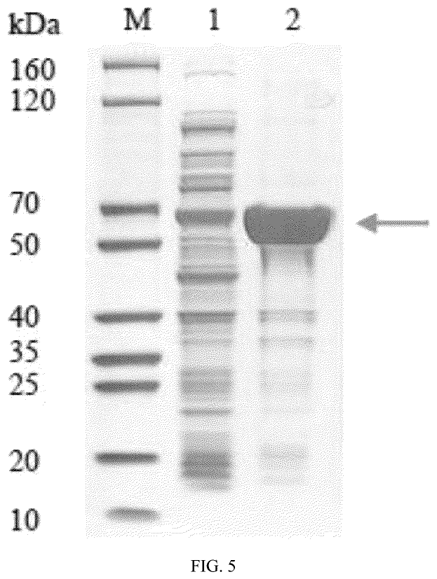

Scale-up culturing was performed. When grown to OD₆₀₀=0.8, IPTG with a final concentration of 0.2 mM was added and induced at 15° C. for 16 h, and then the bacteria were collected. The whole bacteria were ultrasonically lysed with 50 mM Tris (pH 8.0), 300 mM NaCl, 20 mM Imidazole containing 1% Triton X-100, 1 mM DTT, and 1 mM PMSF, and the supernatant was collected, and the supernatant and precipitate were analyzed and detected by SDS-PAGE, as shown in FIG. 5. The arrow indicates the SgTPS7 protein.

### Example 5 Purification of SgTPS7 Protein Encoded by Germacrene Synthase D Gene of Sindora glabra Merr. Ex De Wit

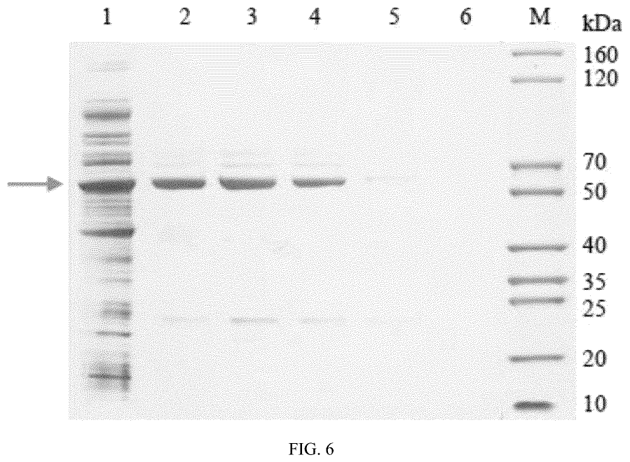

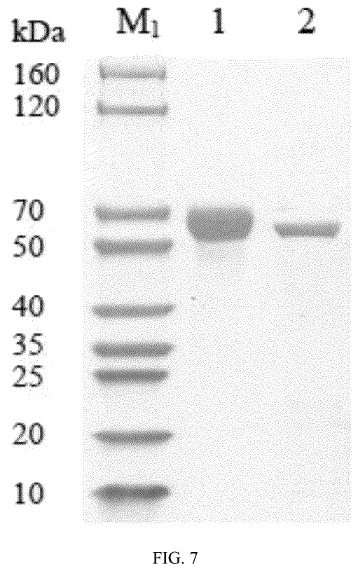

The whole bacteria were ultrasonically lysed with 50 mM Tris (pH 8.0), 300 mM NaCl, 20 mM Imidazole containing 1% Triton X-100, 1 mM DTT, and 1 mM PMSF for 45 min, and the supernatant and precipitate were collected respectively after centrifugation. The Ni-IDA affinity chromatography column was equilibrated with 50 mM Tris (pH 8.0), 300 mM NaCl, and 20 mM Imidazole buffer, and the target protein was eluted with equilibration buffers with different imidazole concentration. The elution fractions were analyzed and detected by SDS-PAGE, as shown in FIG. 6, where the arrow indicates the SgTPS7 protein. After purification by Ni-IDA affinity chromatography, the elution fractions with relatively high purity and concentration were collected and dialyzed into 1×PBS (pH 7.4), 5% Glycerol, filtered with a 0.22 μm filter after dialysis was completed, and aliquoted and frozen at −80° C. The protein concentration was determined using a Bradford protein concentration assay kit, as shown in FIG. 7, and the protein concentration of SgTPS7 protein was 0.240 mg/ml.

### Example 6 Biochemical Function of Germacrene Synthase SgTPS7 of Sindora glabra Merr. Ex De Wit

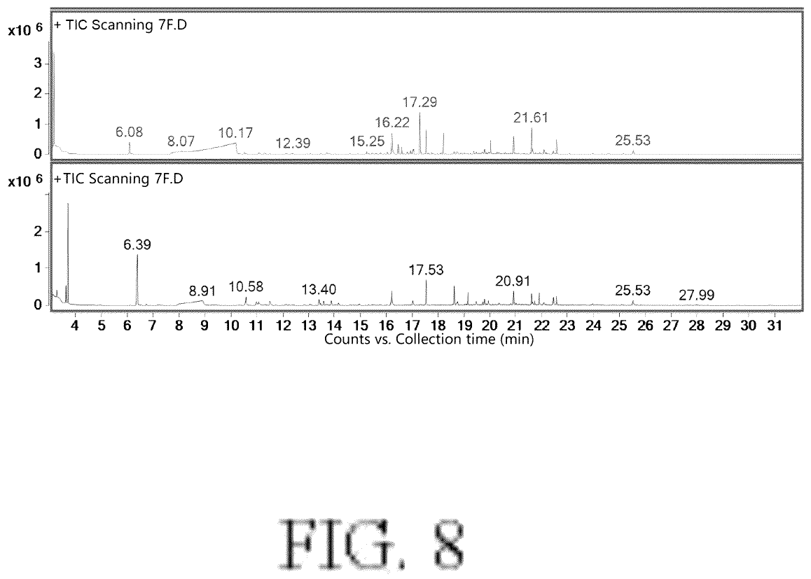

Farnesyl pyrophosphate (FPP) or geranyl pyrophosphate (GPP) was used as the substrate. The enzymatic reaction system included: 25 mM Tris-HCl (pH 7.4), 5 mM dithiothreitol (DTT), 100 mM potassium chloride, 5 mM magnesium chloride and 10% glycerol, and the substrate concentration was 50 μM. 50 μg of purified sesquiterpene synthase SgTPS7 protein of Sindora glabra Merr. ex de Wit was added and placed at 37° C. for reaction for 1 h. After the reaction was completed, headspace extraction of the volatile substances was performed for 30 min with solid phase microextraction SPME fiber PDMS 100 μm, followed by desorption at 250° C. for 3 min and sample injection. The catalytic products were detected by using GC-MS (Agilent GC-MS 7890B-5977A). The gas chromatography column was HP-5MS (30 m×0.25 mm); the flow rate of carrier gas high-purity He was 1.0 mL/min; the temperature rising program included: held at 50° C. for 1 min, heated to 80° C. at a rate of 5° C./min, held for 1 min, then heated to 220° C. at a rate of 10° C./min, and held for 10 min; the temperature of sample injection port was 250° C., the temperature of the ion source EI 70 eV was 230° C.; the interface temperature was 250° C.; the collection mass range was 30-200 amu; and the data were searched through the NIST 14 mass spectrometry library and compared with the standard spectrum to identify the peaks of individual components. The total ion current of the sample is as shown in FIG. 8. When FPP is used as the substrate, peaks appear at retention times of 16.46, 16.60, 17.29, and 18.20, respectively. After searching in the NIST 14 data retrieval library and comparing, the results show that the corresponding compounds are β-Ylangene, β-Copaene, Germacrene D, and Nerolidol, as shown in FIG. 8, among which Germacrene D is the main compound. When GPP is used as the substrate, a peak appears at a retention time of 10.98. After searching in the NIST 14 data retrieval library and comparing, the results show that the corresponding compound is Linalool, as shown in FIG. 8. This indicates that the sesquiterpene synthase SgTPS7 of Sindora glabra Merr. ex de Wit belongs to the germacrene synthase, and can catalyze FPP to synthesize four sesquiterpene compounds, among which germacrene is the main product, and can catalyze GPP to synthesize linalool monoterpene compounds.

The various embodiments in this specification are described in a progressive manner, and each embodiment focuses on the differences from other embodiments. For the same or similar parts between the various embodiments, reference may be made to each other.

The above description of the disclosed embodiments enables one skilled in the art to implement or use the present disclosure. Various modifications to these embodiments will be obvious to one skilled in the art, and the general principles defined herein may be implemented in other embodiments without departing from the spirit or scope of the present disclosure. Therefore, the present disclosure will not be limited to the embodiments shown herein, but rather conform to the widest scope consistent with the principles and novel features disclosed herein.

1. Use of germacrene synthase SgTPS7 protein or a coding gene of SgTPS7 protein in catalyzing FPP to from germacrene D, wherein an amino acid sequence of the SgTPS7 protein is as shown in SEQ ID NO. 1, and a nucleotide sequence of the coding gene is as shown in SEQ ID NO. 2.


SEQUENCE LISTING (4 sequences)

SEQ ID NO: 1 - protein, 548 aa, synthetic construct
  source 1..548: note = SgTPS7 gene encoded protein
MTRSTAGFTPSVWGNQFLSVASSPSMKNKRVEIHQYLQNLKQQLERQLKSVEEPSEKLNLIDTMQRLGVSYHFQSEIEESLKHLHKNPPPSWNAKDIDSHLLGIALWFRLLRQQGYYVSCDIFNKFKDEKGDFKTTLINDVEGMLALYEAAYLGIRGEEILDQMLEFTTSYLKSRLNDMTPYLQERTNRALYCPIHKGLPKIETRYYISMYSKKDSRNDLLLEFAILDFNILQQQYQKELSYITEWFKKLDFVSKVPYTRDRIVEGYFWPLGAYFEKQYSKGRRIVAKIIGAFSSLDDTYDNFGTVDELNVFTEAIMRWDINLVASLPESMKVVFENILNLLIEIELLTEEDEISFAVEYVKQGIQGLVTGYMLEAEWRGRGYIPTYEDYIANGIWTAGYPALEIISLLGLGNIATREVFDWISSMPKIVRASGIVGRIGNDLASHKREQNSGHVATSVECYMKQYGISEDEAYKLLLKEIENAWKDLNEEYMKPNGIPKVVLERVRNFSRASEFFYGRFVDNYTNGERLKDHIAALFLDPIAIDHNK

SEQ ID NO: 2 - DNA, 1647 bp, synthetic construct
  source 1..1647: mol_type = other DNA; note = nucleotide of the SgTPS7 gene
atgactagatccacagcaggcttcactcctagtgtttggggaaatcagttcctttccgttgcttccagcccctcgatgaagaacaaaagagtggaaattcatcaatatttacagaatctgaaacaacaactggagagacagctaaaaagtgttgaagagccctctgaaaaattaaacttgatcgataccatgcaacgtttaggagtgtcttaccattttcaaagtgagatcgaagaatcattaaaacatctgcataagaatcctcctccttcctggaatgccaaagatatcgattctcatcttttgggcattgctctttggtttcggctgcttagacaacaaggctattacgtatcatgcgatatatttaacaaatttaaagatgagaaaggtgatttcaagacaacattgatcaatgatgtggaaggaatgctagccttatatgaagctgcatatctcggtatccgtggagaagaaattcttgatcaaatgctagaatttacaacgtcttaccttaaatcaaggttgaatgacatgacaccctaccttcaagaaagaacaaatcgtgctttgtactgtcctatacacaaaggcttaccaaaaattgagaccagatattacatttctatgtattcaaaaaaggattctcggaatgatctgctactagaatttgcgatactagatttcaatatcttgcaacaacaatatcagaaggaattaagctacatcacagagtggtttaaaaaattagatttcgtgagcaaggttccttacaccagagacagaattgttgaaggctacttttggcctttgggagcatactttgagaagcaatatagcaaaggaagaagaattgtggccaaaattattggtgctttttcatctttggatgatacttatgataacttcggcacagtggatgaactcaatgtctttactgaagcaattatgagatgggatattaatctagtagcttctctccctgaatccatgaaagtggtatttgaaaatattttaaatttgttgattgaaatagagttgttaaccgaagaggacgaaatatcctttgctgttgaatatgttaaacaagggattcaaggtttagtgacaggatacatgcttgaagctgagtggaggggcagaggctacataccaacatatgaggactacatagcgaatggaatttggaccgctggttacccagcccttgaaataatatccttacttggtctcggaaatatcgcaacccgagaggtgtttgattggatttctagtatgcctaaaattgttagagcttcaggaattgtgggcagaataggaaatgatttggcttctcataagagggaacaaaatagcggacatgttgcaacttcagttgagtgttacatgaagcaatatgggatttcagaagatgaagcctataaactactacttaaggagatagagaatgcatggaaagatcttaacgaagagtatatgaagccaaatggtataccaaaggtggtacttgagcgtgtacggaatttctcgcgtgcaagtgagtttttctatggtcgctttgttgataattacacaaatggagagaggctgaaggatcacattgctgcactttttctggatcccatagcaattgatcacaataaatag

SEQ ID NO: 3 - DNA, 1681 bp, synthetic construct
  source 1..1681: mol_type = other DNA; note = base sequence of SgTPS7 fusion protein
catatgcatcatcatcatcatcacggtcgtccgaccgcaaattttagtccgagcgtttggggcaaccagtttttatctattgcgagcggtccgctgctgaaaaacaaagaagcggagatccaccagcatctgctgaacctgaaagaacaggtccgcaaacagctgaaaaacggcgttgaagaaccgagcgagaaactgaacctgatcgataccatccaacgtctgggcgttagccatcatttcgaacgcgaaatcgaagagagcctgaaacatctgcataaaaacccgccgagtagttggaacgcagaagatatcgacgcgcatctgctgtctatcagtctgtggtttcgtctgttacgtcagcagggctattacgttagctgcgacgttttcgacaaattcaaagacgacaaaggcatgttcaaaaccaccctgattgacgacgtcgaaggtatgttagcgctgtacgaagcaacctatctgggtattcgcggcgaagaaatcctggatcaggttctggagttcaccatgttccacctgaaaagccgtctggaaggcatgaccccgtatttacgcgaacgcgcagatcgcgcattatattgtccgatcaacaaaggcctgccgcgtattgaaacccgctacttcatcagcatgtacagcaaaaaagacagccgtaacgatctgctgctggaatttgcaatgctggactttaacatcctgcagcagcagtaccagaaagaactgtctcacctgagcgagtggtacaaaaaactggacttcgtcagcaaagtcccgtatacccgcgatcgtattgtcgaaggctatttctggccgttaggcgcatatttcgagaaccagtacaacaaaggccgcatcatcgtcagcaaactgatcctggttctgaccgcattagacgatacctacgacgcatacggtaccgttgacgaactgaaactgttcaccgaggcgattaaacgctgggacatcaacatggtcgcttctttaccggagtgcatgaaagtcgtctttcaggcgatcctggacttactggacgaaatggaactgctgaccgaagctgacggtattagctgcttcgtcgaatacgttaaaccggcactgcaggatctggctaaaagctatctgctggaagcagagtggcgcgataaaggttacatcccgacctacgaagaatatatcgcgaacggcgtttttagctgcggttatccggccgttgaaatggcaagtctgctgggtctgggcaaaattgcgaccaaagaagtcttcgattggatcagcaacgttccgaaaattgttcgcgcgtctagcatcatgtgccgtttaaccgacgatctggcaagccacaaattcgaacagaaccgcgaacacgtcggttccgctatcgagtgctacatgaaacagtacggcgttagcgaagaagaagcctataaaatgctgctgaaagagatcgagaacggctggaaagacctgaacgaagaatatatgaaaccgaacggcgttccgaaagttgtactgaaatgcgtcctgaacttcagccgcgttattgaattcctgtacggccacttcgtcgacaaatacaccaacgcggaaatgctgaaagaccacatcgcgagcctgtttgttgatccgattgccattgacgtcaacaaataatgaaagcttg

SEQ ID NO: 4 - protein, 554 aa, synthetic construct
  source 1..554: note = SgTPS7 fusion protein
MHHHHHHTRSTAGFTPSVWGNQFLSVASSPSMKNKRVEIHQYLQNLKQQLERQLKSVEEPSEKLNLIDTMQRLGVSYHFQSEIEESLKHLHKNPPPSWNAKDIDSHLLGIALWFRLLRQQGYYVSCDIFNKFKDEKGDFKTTLINDVEGMLALYEAAYLGIRGEEILDQMLEFTTSYLKSRLNDMTPYLQERTNRALYCPIHKGLPKIETRYYISMYSKKDSRNDLLLEFAILDFNILQQQYQKELSYITEWFKKLDFVSKVPYTRDRIVEGYFWPLGAYFEKQYSKGRRIVAKIIGAFSSLDDTYDNFGTVDELNVFTEAIMRWDINLVASLPESMKVVFENILNLLIEIELLTEEDEISFAVEYVKQGIQGLVTGYMLEAEWRGRGYIPTYEDYIANGIWTAGYPALEIISLLGLGNIATREVFDWISSMPKIVRASGIVGRIGNDLASHKREQNSGHVATSVECYMKQYGISEDEAYKLLLKEIENAWKDLNEEYMKPNGIPKVVLERVRNFSRASEFFYGRFVDNYTNGERLKDHIAALFLDPIAIDHNK